(19) United States
(12) Patent Application Publication
(10) Pub. No.: US 2007/0260243 A1
(43) Pub. Date: Nov. 8, 2007

(54) Bone Correction Device

(76) Inventor: Kazuo Kagami, Toyama (JP)

Correspondence Address: FLYNN THIEL BOUTELL & TANIS, P.C., 2026 RAMBLING ROAD, KALAMAZOO, MI, 49008-1631, US

(21) Appl. No.: 11/631,494

(22) PCT Filed: Nov. 2, 2004
(86) PCT No.: PCT/JP04/16253

§ 371 Date: Jan. 3, 2007

(51) Int. Cl. A61B 17/60

(52) U.S. Cl. 606/57; 606/54

(57) ABSTRACT

There is provided a tower-shaped frame provided to stand upright on a positionable platform, a bone correction rod held by an elevating supporter, wherein the elevating supporter is mounted on the tower-shaped frame to be adjustably movable up and down, a bone correction rod, and a weight receiver Suspended from the elevating supporter to be movable up and down so as to apply a load of weights to the bone correction element as a bone correction force. When the bone correction device is used, a height of the elevating supporter is adjusted depending on a size, a posture of a body of a patent and a position of the patient, and so forth, and a necessary weight is placed on the weight receiver depending on the symptom of the patient and so forth. When the bone correction element is applied to an affected part, a pressing for is applied to the affected part in proportion to the weights, and hence if the pressing force is continuously applied to the affected part, an obstinate strain of a physique can be easily corrected.

### TECHNICAL FIELD

The present invention relates to a bone correction device that is used mainly for commercial purpose and applies a bone correction element such as a spherical body and so forth to affected part instead of fingers of an operator so as to correct strain or slippage of cervical vertebrae, pelvis, skull, and so forth of a human body.

### BACKGROUND TECHNOLOGY

All illness or disease such as backache, stiffness in the shoulder, disease of an internal organ, skin disease and so forth are frequently caused by strain of a bone structure. Cause and effect relationship between the bone structure and other organs such as circulatory organ, endocrine organ, respiratory organ has not learned in modern medical science but there is a history in which it has been studied in oriental medicine for a long period of time. According to the oriental medicine, the strain of the bone structure can be corrected by mainly hands, particularly by thumbs.

Bone correction technique is based on a concept that a human body can be recovered from all disease by correcting slippage of bones about the backbone or pelvis upon grasping a human body structurally. According to this concept, it is not unusual to correct the bone structure by one or two times operation, but there is a case that the slippage or strain of the bone is stiff because it is caused for ten years or twenty years. It seems that this is caused by the fact that the slippage or strain of the body influences the level of the cells of the body.

The applicant figured out that the portion to be remedied has to be continuously corrected for several ten minutes so as to release abnormal information of the cells of the entire body. However, it is hardly possible to continuously correct the bone by applying a constant force ranging from 3 kg to 4 kg by hands. There comes a limit of continuous application of the constant force for 10 to 20 seconds even for a professional.

### PROBLEMS TO BE SOLVED BY THE INVENTION

The present invention has been made based on the foregoing concept, and it is an object of the present invention to provide a bone correction device capable of continuously applying a strong force to a portion of a body to be pressed and also capable of not only optionally selecting the portion to be pressed within a predetermined range but also easily adjusting quantity and direction of the force.

### MEANS FOR SOLVING THE PROBLEMS

To solve the above-mentioned problem, the present invention is to provide a bone correction device comprising a tower-shaped frame provided to stand upright on a positionable platform; a bone correction rod held by an elevating supporter, said elevating supporter being mounted on the tower-shaped frame to be adjustably movable up and down; a bone correction element provided on an end of the bone correction rod; and a weight receiver suspended from the elevating supporter to be movable up and down so as to apply a load of weights to the bone correction element as a bone correction force.

With the bone correction device being structured as mentioned above, when this is used, a height of the elevating supporter is adjusted depending on a size, a posture of a body of a patient, and a position of the affected part, and so forth, and a necessary weight is placed on the weight receiver depending on the symptom of the patient and so forth. When the bone correction element is applied to the affected part, a pressing force is applied to the affected part in proportion to the weight, and hence if the pressing force is continuously applied to the affected part, an obstinate strain of a physique can be also easily corrected.

If the tower-shaped frame comprise a pair of forked vertical rails (Claims 2 and 3), it can be moved while the upper ends of the forked vertical rails are caught by the hands and made easy in handling. Further, if the weight receiver is suspended by a wire (Claim 2 and succeeding claims), a force of the weight can be easily applied to the bone correction element with a simple structure. Still further, there is a case where the bone correction element operates to be pushed downward by a slide holding device of the bone correction element provided on the elevating supporter (Claim 2 and Claim 4), or operates to be pushed in a straight forward direction (Claims 3, 4)

For the bone correction element, if it is formed of pure copper or copper lump of a high degree of purity covered with a plating made of an alloy of nickel and rhodium (Clams 5, particularly in the case of Claim 6), a body current can be promoted compared with the pure copper owing to the presence of the plating, so that the effect of bone correction can be further promoted by synergy of pressing force.

### EFFECT OF THE INVENTION

As explained above, according to the bone correction device of the present invention, it is possible to continuously apply a strong force to a part to be corrected serving as an affected part of the body by the quantity of the weight, to optionally select the part to be corrected by adjusting the height position of the elevating supporter, and also to adjust the quantity and direction of the force with ease, there is such an excellent effect that a stubborn strain or slippage of the physical structure caused by life habit can be corrected with ease while taking time.

### BEST MODE FOR CARRYING OUT THE INVENTION

Although any of the attached drawings shows the embodiment of the present invention (Claim 1), more in detail, FIG. 1 corresponds to Claim 2, FIG. 2 corresponds to Claim 3, FIG. 3 corresponds to Claim 4, and FIG. 4 corresponds to Claims 5 and 6.

The bone correction device according to Claim 2 comprises, as shown in FIG. 1, a tower-shaped frame 5 is provided on a platform 1 and formed of a pair of left and right forked vertical rails 5a, 5a, an elevating supporter 6 to which a bone correction rod 7 is attached and a weight receiver 9 which is suspended from the elevating supporter 6 by a wire 11, wherein the elevating supporter 6 and the weight receiver 9 are held on the tower-shaped frame 5 to be vertically movable between the vertical rails 5a, 5a serving as a guide.

A guide rod 8 is provided on the platform 1 to be adjustable in its projection length in order to position the platform 1 with ease. The guide rod 8 has an engaging part 16 so as to engage with a guide rail 2 disposed around a finger pressure bed and a screw 19 for pressing and fixing the guide rod 8 is provided on the platform 1. Casters 15, 15, . . . are mounted on the platform 1 at four corners thereof.

Since the pair of left and right vertical rails 5a, 5a constituents of the tower-shaped frame 5 are inverted in the reverse U-shape at the upper ends thereof and the portions under the upper ends are parallel with each other, they can be handled with ease while held by hand. The lower ends of the vertical rails 5a, 5a are fixed to the platform 1, and connection plates 17, 17 are screwed on the upper ends of the vertical rails 5a, 5a at the front and back thereof in the vicinity of the reverse U-shaped portions. Connection plates 18, 18 are screwed on the middle portion of the vertical rails 5a, 5a, and an intermediate plate 21 is extended between and fixed to the connection plates 18, 18 by screws. The elevating supporter 6 is opened in its opposite sides so as to be movable between the connection plates 17, 17 and the intermediate plate 21, and the intermediate plate 21 has a through hole 23 at the center thereof through which the wire 11 suspended from the elevating supporter 6 passes.

The elevating supporter 6 comprises a guide block 6a which rises and falls between the vertical rails 5a, 5a at the back side as a guide (in a direction of P) and a slide holder 6b which is pivotally supported by the guide block 6a with fulcrum pins 24 wherein the guide block 6a is fastened by a male screw 22 which is screwed in a pair of female screw plates 20 of the guide block 6a, and it is fixed to the vertical rails 5a, 5a while being clamped by the female screw plates 20. Meanwhile, the slide holder 6b has a block-shaped main body and is fixed to the guide block 6a by the fulcrum pin 24 and to which a slide rail 28 is integrally fixed. The slide rail 28 holds the bone correction rod 7 at the lower surface thereof while slidably brought into contact therewith, and has an end face plate 30 fixed at the tip end through which the bone correction rod 7 penetrates and held.

The bone correction rod 7 is slidably penetrated through the main body of the slide holder 6b as well as the end face plate 30 so as to slide on the lower surface of the slide rail 28, while a screw 34 is provided on the main body of the slide holder 6b so as to push the bone correction rod 7 against and fix the same to the slide holder 6b, and a wire 11 is connected to the end face plate 30 which is always positioned at the center thereof. A bone correction element 35 is fixed to a fixing terminal 36 provided on the front end of the bone correction rod 7 by a screw (see FIG. 4). With the arrangement set forth above, the bone correction rod 7 can be adjusted in a height (in a direction of P), a length (in a direction of R) and an angular (in a direction of S) so that the bone correction element 35 is pertinently used so as to comfortably apply to the affected part of a body B.

The weight receiver 9 is formed of a metal plate having a rectangular frame shape which is opened at the left and right so that plate-shaped weights 10 can be put in and out from these openings, and it is provided vertically on an engaging plate 37 which engages with the tower-shaped frame 5, and a connection metal fitting 40 which can adjust the length of the wire 11 is fitted to the center of the engaging plate 37. Further, both ends 44, 44 of the weight receiver 9 can engage with the tower-shaped frame 5, and the weight receiver 9 moves up and down vertically (in a direction of Q) in response to the swinging motion of the bone correction rod 7 while being suspended by the wire 11.

The bone correction device as set forth in Claim 3 in structured, as shown in FIG. 2, substantially in the same manner as described in the foregoing embodiment, such that a reverse U-shaped tower-shaped frame 5 which is formed of vertical rails 5a, 5a and stands upright on a platform 1. Accordingly, the constituents of the bone correction device are omitted in explanation while the same reference numerals are used. This embodiment is mainly different from the foregoing embodiment in respect of an elevating supporter 6 and a bone correction rod 7 fixed to the elevating supporter 6, and so forth.

The elevating supporter 6 comprises a guide block 6a and a slide holder 6b wherein the slide holder 6b is formed of a longish square cylinder through which a bone correction rod 7 passes, and it is clamped by the vertical rails 5a, 5a at both ends and slightly protruded outward and fixed to the guide block 6a so as to move up and down horizontally. A bone correction element 35 is detachably fitted to the front end of the bone correction rod 7 by the screw. The bone correction element 35 is further fitted to the back side of the bone correction rod 7 and a long bolt screw 38 is fitted to the bone correction element 35 and it is suspended from the bone correction rod 7.

Since a pulley 45 hooking wire 11 is fitted to the middle of the slide holder 6b, and a connection metal fitting 47 for holding the wire 11 is fitted to the back end of the bone correction rod 7 so as to adjust the length of the wire 11, a load of weights 10 placed on a weight receiver 9 pushes the bone correction rod 7 forward, thereby generating a pushing force by which the bone correction element 35 is pressed against the body B.

The bone correction device as set forth in Claim 4 comprises, as shown in FIG. 3, a tower-shaped frame 5 which stands upright on a platform 1, and which is structured by a pair of straight left and right struts 5b, 5b, an elevating supporter 6 mounted on the tower-shaped frame 5 and a bone correction rod 7 fixed to the elevating supporter 6 so as to move slidably and swingably.

The elevating supporter 6 comprises guide blocks 6a, 6a which are inserted into the struts 5b, 5b, respectively, and a cylindrical slide holder 6b which is pivotally swingably supported between the guide blocks 6a, 6a, while bone correction elements 35, 35 are fitted to both ends of a bone correction rod 7 via shafts of male screws 49, 49. Although the bone correction rod 7 is swung up and down about a rotary axis (not sown) of the bearing metal fitting 51 integrated with the slide holder 6b, a wire 11 is hooked by the rotary axis of the bearing metal fitting 51 and a connection metal fitting 47 for hooking the wire 11 is fitted to the rear end of the bone correction rod 7, so that the bone correction rod 7 is pushed forward (see the arrow R) by a load of weights 10 placed on a weight receiver 9 in the same manner as the embodiment shown in FIG. 2.

A fitting piece 43 is suspended from the rear end of the bone correction rod 7 so as to fit the connection metal fitting 47 thereto for pushing the bone correction rod 7 forward. Further, since a fitting piece 48 for hooking the connection metal fitting 47 is suspended from the front end of the slide holder 6b, when the wire 11 is also hooked by the connection metal fitting 48, the bone correction rod 7 is pushed downward (In a direction of S). Meanwhile, a male screw 53 for stopping the movement of the bone correction rod 7 at the time of non-use thereof is fitted on the slide holder 6b. Other components are the same as those set forth in the previous embodiments, and hence they are depicted by the same reference numerals and the explanation thereof is omitted.

In any of the foregoing embodiments, a metallic contact for promoting the growth of the body current is employed for the bone correction element 35 (Claims 5 and 6). This is explained with reference to FIGS. 4 and 5.

With a bone correction element 35 serving as a metallic contact for promotion of the body current, a copper lump 55 of pure copper is plated with an alloy of nickel (Ni), and rhodium (Rh), and a plating 57 of the alloy is deposited on the surface of the copper lump 55. For a copper lump 1, use is made of a lump formed in a spherical shape by abrading a lump of 100% pure copper, and as a result of various tests using background spectrum analyses of the most suitable material, it was found out that an alloy of a plating 3 had composition of 53.07 wt. % (at. %: 66.466) of nickel, and 46.93 wt. % (at. %: 33.534) of rhodium. FIG. 5 shows the results of the analyses.

As for pure copper, there are available methods of producing oxygen-free copper, tough pitch copper, and phosphorous-deoxidized copper, and use may be made of pure copper produced by any of those methods, however, the oxygen-free copper is obtained by lowering oxygen content by means of deoxidation in a reducing atmosphere or vacuum fusion, so that there is no risk of occurrence of hydrogen embrittlement, and it has few impurity elements. Accordingly, pure copper produced by the method of producing the oxygen-free copper was adopted as optimum for use.

However, the tough pitch copper is preferably used in promotion of the body current because it is excellent in electroconductivity, and thermal conductivity. Furthermore, because the tough pitch copper is excellent in malleability, drawing workability, and weather resistance, the same is suitable for fabrication of the metallic contact not only simply in the shape of a sphere but also in the complicated shape of a personal ornament such as an earring, and so forth. However, the tough pitch copper has a drawback in that if it is heated in a reducing atmosphere, there is the risk of occurrence of hydrogen embitterment. Meanwhile, with the phosphorous-deoxidized copper, oxygen content is lowered with the use of phosphorous as a deoxidant, so that there is no risk of occurrence of hydrogen embitterment, however, the same has a drawback in that its conductivity is slightly lower than that of the tough pitch copper.

### BRIEF DESCRIPTION OF DRAWINGS

FIG. 1 is a perspective view showing a bone correction device according to an embodiment of the present invention;

FIG. 2 is a perspective view showing a bone correction device according to another embodiment of the present invention;

FIG. 3 is a perspective view showing a bone correction device according to still another embodiment of the present invention;

FIG. 4 is a sectional view showing the bone correction device according to the present invention; and

FIG. 5 shows results of the spectrum analyses detecting metallic composition of a plating in the bone correction device for promoting a body current according to the embodiment of the present invention.

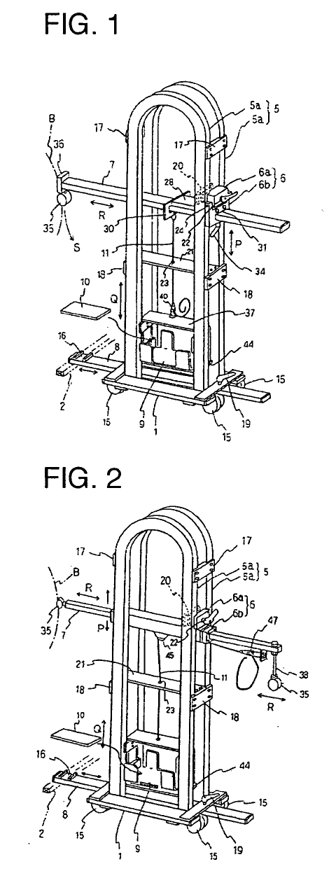

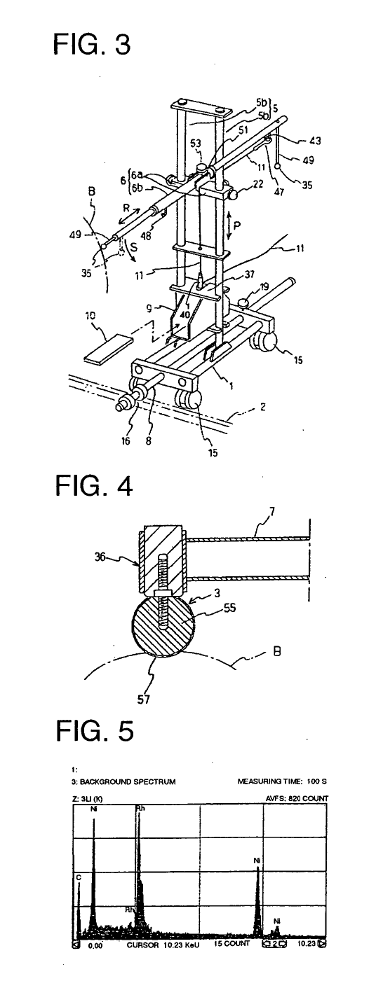

Explanation of Numerals:

### EXPLANATION OF REFERENCE NUMERALS

1 platform
5 tower-shaped frame
5a forked vertical rail
5b strut
6 elevating supporter
6a guide block
7 bone correction rod
9 weight receiver
10 weights
11 wire
35 bone correction element
47 connection metal
55 copper lump
57 plated layer

1. A bone correction device comprising a tower-shaped frame provided to stand upright on a positionable platform; a bone correction rod held by an elevating supporter, said elevating supporter being mounted on the tower-shaped frame to be adjustably movable up and down; a bone correction element provided on an end of the bone correction rod; and a weight receiver suspended from the elevating supporter to be movable up and down so as to apply a load of weights to the bone correction element as a bone correction force, wherein the tower-shaped frame comprises a pair of forked vertical rails reversed at upper ends thereof, and spaced in parallel with each other at the left and right so as to serve as a guide of the elevating supporter and the weight receiver, and wherein the bone correction rod is clamped by the pair of forked vertical rails and protruded longitudinally from both sides of the forked vertical rails, and the elevating supporter for supporting the bone correction rod is held by the rear ends of the forked vertical rails so as to be adjustable up and down.

2. A bone correction device according to claim 1, wherein the elevating supporter is provided with a slide holder for holding the bone correction rod to be swingable up and down, and the weight receiver is suspended from the slide holder of the elevating supporter by a wire so as to apply a down-pressing force to the bone correction element by a load of the weights.

3. A bone correction device according to claim 1, wherein the elevating supporter is provided with a slide holder for holding the bone correction rod, and the slide holder is provided with a pulley from which the wire is hooked so as to apply a biasing force to the bone correction rod in a sliding direction by the weights, and a connection metal fitting is fitted to one end or both ends of the bone correction rod so as to suspend the weight receiver by the wire.

4. A bone correction device comprising a tower-shaped frame provided to stand upright on a positional platform; a bone correction rod held by an elevating supporter, said elevating supporter being mounted on the tower-shaped frame to be adjustably movable up and down; a bone correction element provided on an end of the bone correction rod; and a weight receiver suspended from the elevating supporter to be movable up and down so as to apply a load of weights to the bone correction element as a bone correction force, wherein the tower-shaped frame comprises a pair of vertical struts spaced in parallel with each other at the left and right so as to serve as a guide of the elevating supporter and the weight receiver, and wherein the bone correction rod is protruded longitudinally between both sides of both struts, and the elevating supporter for supporting the bone correction rod is held by both struts so as to be adjustable up and down when inserted between both struts, wherein the elevating supporter pivotally swingably supports a slide holder for holding the bone correction rod, and a connection metal fitting for hooking the wire is fitted to one end or both ends of the bone correction rod so as to suspend the weight receiver by the wire hooked by the slide holder in order to apply a biasing force to the bone correction rod in the sliding direction by the weights.

5. A bone correction device according to claim 1, wherein the bone correction element is formed of pure copper or copper lump of a high degree of purity covered with a plating made of an alloy of nickel and rhodium.

6. A bone correction device according to claim 5, wherein copper lump contains 95 wt. % or more of copper, and the alloy of nickel and rhodium contains 40 to 60 wt. % of nickel and 40 to 60 wt. % of rhodium.
